(19) United States
(12) Patent Application Publication
(10) Pub. No.: US 2017/0320047 A1
(43) Pub. Date: Nov. 9, 2017

(54) PROCESS TO PREPARE A COMPOSITE IONIC LIQUID

(71) Applicants: SHELL OIL COMPANY, HOUSTON, TX (US); SHELL INTERNATIONALE RESEARCH MAATSCHAPPIJ B.V., The Hague (NL); CHINA UNIVERSITY OF PETROLEUM (BEIJING), Beijing (CN)

(72) Inventors: Rui ZHANG, Changping, Beijing (CN); Zhichang LIU, Changping, Beijing (CN); Xianghai MENG, Changping, Beijing (CN); Chunming XU, Changping, Beijing (CN)

(21) Appl. No.: 15/520,484

(22) PCT Filed: Oct. 22, 2015
(86) PCT No.: PCT/EP2015/074475

§ 371 Date: Apr. 20, 2017

(30) Foreign Application Priority Data
Oct. 23, 2014 (CN) PCT/CN2014/089289

(51) Int. Cl. B01J 31/02; B01J 31/30; C07C 2/60

(52) U.S. Cl. CPC B01J 31/0279; C07C 2/60; B01J 31/30; B01J 2231/32; C07C 2531/02

(57) ABSTRACT

The present invention provides a process to prepare a composite ionic liquid, the process at least comprising the steps: (a) mixing an ammonium salt and a solid aluminium salt to obtain a first mixture; (b) stirring under heating the first mixture of step (a); (c) adding to the first mixture of step (b) one or more solid metal salts to obtain a second mixture, wherein the metal salts are selected from halides, sulfates, or nitrates of aluminium, gallium, copper, iron, zinc, nickel, cobalt, molybdenum and platinum; (d) stirring under heating the second mixture of step (c); (e) adding to the second mixture of step (d) a hydrocarbon to obtain a third mixture; (f) stirring under heating the third mixture of step (e) until the solids of the aluminium salt of step (a), and the solids of the metal salts of step (c) disappear and the mixture is converted into a composite ionic liquid; and (g) cooling the composite ionic liquid of step (f).

The present invention provides a process to prepare a composite ionic liquid and a composite ionic liquid obtainable by this process. The present invention further provides a process for preparing alkylate using said composite ionic liquid.

There is an increasing demand for alkylate fuel blending feedstock. As a fuel-blending component alkylate combines a low vapour pressure, no sulfur, olefins or aromatics with high octane properties. The most desirable components in the alkylate are trimethylpentanes (TMPs), which have research octane numbers (RONs) of greater than 100. Such an alkylate component may be produced by reacting isobutane with a butene or a mixture of butenes in the presence of a suitable acidic catalyst, e.g. HF or sulfuric acid, although other catalysts such a solid acid catalyst have been reported. Recently, the alkylation of isoparaffins with olefins using an acidic ionic liquid catalyst has been proposed as an alternative to HF and sulfuric acid catalysed alkylation processes.

For instance, U.S. Pat. No. 7,285,698 discloses a process for manufacturing an alkylate oil, which uses a composite ionic liquid catalyst to react isobutane with a butene.

In the process of U.S. Pat. No. 7,285,698, isobutane and a butene are supplied to a reactor and the alkylate is formed by contacting the reactants with a composite ionic liquid under alkylation conditions. The reactor effluent is separated and the ionic liquid phase is recycled to the reactor while the hydrocarbon phase is treated to retrieve the alkylate. The copper(I)chloride (CuCl) or silver(I)chloride (AgCl) content of the composite ionic liquid results in very good selectivity of alkylation. However, the presence of CuCl or AgCl in the composite ionic liquid results in the formation of solids during operation of such an ionic liquid alkylation process. As the reaction progresses, these solids accumulate in the recycled ionic liquid phase and may lead to blockage of pathways and/or valves. The relation between the presence of CuCl in the composite ionic liquid and the formation of solid during composite ionic liquid catalysed isobutene alkylation is for example described in Energy Fuels, 2014, 28 (8), pp 5389-5395.

In WO2011/015639 a process is described for removal of the solids formed during the ionic liquid alkylation process. According to that process, a solids-comprising effluent comprising hydrocarbons and acidic ionic liquid is withdrawn from the reaction zone and at least part of the solids-comprising effluent is treated to remove at least part of the solids to obtain a solids-depleted effluent. It has however been found that solids removal according to the process of WO2011/015639 is difficult because of high viscosity of the ionic liquid. Centrifugation of the solids-comprising effluent is therefore complex and is accompanied by high energy consumption. Filtration is not very practical because it is time consuming and requires high pressures. Finally, settling is even more time consuming and therefore not a desirable solution.

It is an object of the invention to solve or minimize at least one of the above problems. It is a further object to provide a more efficient method for preparation of alkylates without or minimize the formation of solids. Another object of the invention is to minimize or avoid the use of CuCl or AgCl in the catalyst preparation.

One of the above or other objects may be achieved according to the present invention by providing a process to prepare a composite ionic liquid, the process at least comprising the steps:
(a) mixing an ammonium salt and a solid aluminium salt to obtain a first mixture;
(b) stirring under heating the first mixture of step (a);
(c) adding to the first mixture of step (b) one or more solid metal salts to obtain a second mixture, wherein the metal compounds are selected from halides, sulfates, or nitrates of aluminium, gallium, copper, iron, zinc, nickel, cobalt, molybdenum and platinum;
(d) stirring under heating the second mixture of step (c);
(e) adding to the second mixture of step (d) a hydrocarbon to obtain a third mixture;
(f) stirring under heating the third mixture of step (e) until the solids of the aluminium compound of step (a) and the solids of the metal compounds of step (c) disappear and the mixture is converted into a composite ionic liquid; and
(g) cooling the composite liquid of step (f).

It has now surprisingly found according to the present invention that by adding hydrocarbons to the reaction mixture during the preparation of the composite ionic liquid reduced lower amount of CuCl or AgCl can be applied which does not go at the expense of the selectivity of the alkylation reaction.

Another advantage of the present invention is that the formation of solids during the alkylation process is reduced or even avoided.

The invention further provides a composite ionic liquid obtainable by said process.

An advantage of the use of said composite ionic liquid is that the formation of solids during alkylation is reduced but still high selectivity to trimethylpentane and dimethylpentane products is achieved.

In step (a) of the process according to the present invention an ammonium salt and a solid aluminium salt and are mixed to obtain a first mixture.

The composite ionic liquid prepared according to the present invention comprises a cation and an anion. The cation in the ammonium salt of step (a) is the cation of the composite ionic liquid.

Suitably, the cations are derived from the hydrohalide or alkylhalide salt of an alkyl-containing amine, imidazolium or pyridine.

Preferably, the cations comprise cations of ammonium salts, for example nitrogen atoms, which are saturated with four substituents, among which there is at least one alkyl group. More preferably, the alkyl substituent is at least one selected from methyl, ethyl, propyl, butyl, pentyl, and hexyl groups.

Examples of preferred ammonium cations include trietylammonium hydrogen (NEt₃H⁺) and methyldiethylammonium hydrogen cations (MeNEt₂H⁺), cations in which the nitrogen is part of a cyclic structure (e.g. like in piperidine, pyrrolidine and 1-alkylimidazole) or

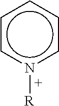

Typically, depending on their structure, ammonium salts are solid or liquid.

Preferably, the ammonium salt in step (a) is a hydrohalide of an alkyl-containing amine, preferably triethylammonium hydrogenchloride (Et₃NHCl).

The solid aluminium salt is the anion of the composite ionic liquid. The anions of the composite ionic liquid are preferably derived from aluminium based Lewis acids, in particular aluminium halides.

Preferably, the aluminium salt in step (a) is an aluminium halide, more preferably aluminium (III) chloride.

The molar ratio of the aluminium salt to the ammonium salt in the composite ionic liquid is preferably from 1.2 to 2.2, more preferably from 1.6 to 2.0, more preferably from 1.7 to 1.9 and most preferably the molar ratio is 1.8.

Suitably, the molar ratio of the aluminium (III) chloride to Et₃NHCl in the composite ionic liquid is from 1.2 to 2.2, more preferably from 1.6 to 2.0, more preferably from 1.7 to 1.9 and most preferably the molar ratio is 1.8.

In step (b) of the process according to the present invention the first mixture of step (a) is stirred under heating.

The first mixture is preferably mixed at a temperature below 100° C., more preferably below 80° C. but preferably above 50° C. Suitably, the mixture is stirred until all solids have converted into the liquid phase.

In step (c) of the process according to the present invention one or more solid metal salts are added to the first mixture of step (c) to obtain a second mixture, wherein the metal salts are selected from halides, sulfates, or nitrates of aluminium, gallium, copper, iron, zinc, nickel, cobalt, molybendium and platinum.

It is preferred to combine the aluminium halide, with a second or more solid metal salt, preferably a metal halide, sulfate or nitrate. By using a coordinate anion comprising aluminium and another metal, an improved alkylate product may be obtained.

Suitable further metal halides, sulfates or nitrates, may be selected from halides, sulfates or nitrates of metals selected from the group consisting of Group IB elements of the Periodic Table, Group IIB elements of the Periodic Table and transition elements of the Periodic Table. Preferred metals include copper, iron, zinc, nickel, cobalt, molybdenum, silver or platinum.

Typically, the metal halides, sulfates or nitrates are primarily metal salts.

Preferably, the metal halides, sulfates or nitrates, are metal halides, more preferably chlorides or bromides, such as copper (I) chloride, copper (II) chloride, nickel (II) chloride, iron (II) chloride.

Further, the solid metal salts added to the first mixture in step (c) are preferably halides of aluminium and copper.

Aluminium(III)chloride and copper(I) chloride are preferably added to the first mixture in step (c).

Typically, the molar ratio of the metal halide of step (c) to the ammonium salt of step (a) in the composite ionic liquid is from 0.1 to 0.5, preferably from 0.1 to 0.4, most preferably from 0.1 to 0.3.

The molar ratio of the copper(I)chloride to Et3NHCl in the composite ionic liquid is suitably from 0.1 to 0.5, preferably from 0.1 to 0.4, more preferably from 0.1 to 0.3.

In step (d) of the process according to the present invention, the second mixture of step (c) is stirred under heating. The second mixture is preferably mixed at a temperature between 100 and 175° C., more preferably between 125 and 150° C., and most preferably between 140 and 150° C. Suitably, the mixture is stirred until all solids have converted into the liquid phase.

In an alternative embodiment of the invention relates to a process for the preparation of a composite ionic liquid, in which process the two or more metal compounds, preferably metal halides are (first) mixed, for instance portion-wise, with the ammonium cations, in the form of an ammonium salt, and (subsequently) the mixture is kept at a temperature of 120 to 170° C. while stirring until all solids have completely converted into the liquid phase.

“Portion-wise” as referred herein means “in at least two portions”. Accordingly, in a portion-wise addition mode, at least (a total of) two portions of the two or more metal halides (e.g. AlCl₃ and CuCl) are added in at least (a total of) two steps to the ammonium salt and mixed with each other. The reaction of the metal halides with the ammonium salt is fast and exothermic. The size of the portions of the metal halides is selected such that the temperature raise is controlled. The mixing time between the addition of the first portion of metal halide and the addition of a subsequent portion is dependent on the nature of the exothermic effect of the addition of the metal halide.

The temperature after addition and mixing of a portion of a metal halide into the ammonium salt or ammonium salt mixture, the latter comprising the ammonium salt and one or more portions of the two or more metal halides, should preferably be kept such that the reactor pressure is higher than the vapour pressure of the aluminium halide at the given temperature. Thus at atmospheric pressure and using aluminium chloride as the aluminium halide the temperature should be kept below 180° C. and preferably below 160° C. to avoid loss of aluminium chloride.

It is noted here that the mixing of the two or more metal salts in this process is not limited to the portion-wise addition mode. Any method to add the metal salts in a manner that controls the heat production may be suitable. Thus, any technical options known in the art for controlled continuous dosing of solids may be applied.

In step (e) of the process according to the present invention, a hydrocarbon is added to the second mixture of step (d) to obtain a third mixture.

Suitable hydrocarbons to be added to the second mixture, are saturated hydrocarbons, unsaturated hydrocarbons and mixtures thereof.

Preferred saturated hydrocarbons are paraffins and cycloalkanes.

Suitable unsaturated hydrocarbons are olefins, cycloolefins, and aromatics. Typically, an olefin added in step (e) of the present invention is dodecene. Also, cyclopentene and cyclohexene are preferred cycloolefins. Further, toluene is a suitable aromatic.

Mixtures of saturated and unsaturated hydrocarbons are preferably coker gasoline and FCC gasoline.

More preferred hydrocarbons added in step (e) of the present invention are coker gasoline and toluene.

Preferably, the amount of hydrocarbon added to the second mixture in step (e) is in the range of 0.5 to 10 mL per 1 mol of ammonium salt, more preferably 1 to 7 mL per 1 mol ammonium salt, and most preferably in the range of 1 to 5 mL per 1 mol of ammonium salt.

Preferably, the amount of hydrocarbon added to the second mixture in step (e) is in the range of 0.5 to 10 mL per 1 mol of Et₃NHCl, more preferably 1 to 7 mL per 1 mol Et₃NHCl, and most preferably in the range of 1 to 5 mL per 1 mol of Et₃NHCl.

Further, the hydrocarbon added to the second mixture in step (e) is preferably toluene or coker gasoline.

In step (f) of the process according to the present invention the third mixture of step (e) is stirred under heating until the solids of the aluminium salt of step (a) and the solids of the metal salts of step (c) completely disappear and the mixture is converted to composite ionic liquid.

The temperature at which the third mixture is stirred in step (f) should preferably be kept such that the reactor pressure is higher than the vapour pressure of the aluminium halide at the given temperature. Thus at atmospheric pressure and using aluminium chloride as the aluminium halide the temperature should be kept below 180° C., preferably below 160° C., more preferably between 120 to 160° C. to avoid loss of aluminium chloride. Also, the temperature is preferably kept such that the reactor pressure is higher than the vapour pressure of the hydrocarbon added in step (e) at a the given temperature.

The solids present in step (f) are salts added in steps (a) and (c).

In step (g) of the process according to the present invention the composite ionic liquid of step (f) is cooled to obtain a cooled composite ionic liquid.

The temperature at which the liquid in step (g) is cooled is at ambient temperature.

A further aspect of the present invention provides a composite ionic liquid obtainable by the process according to the present invention.

In another aspect the present invention provides a process to prepare an alkylate product, the process at least comprising the steps:

(aa) providing a hydrocarbon mixture comprising at least an isoparaffin and an olefin;
(bb) subjecting the mixture of step (aa) to an alkylation reaction, wherein the hydrocarbon mixture is reacted with an composite ionic liquid according to the present invention to obtain an effluent comprising at least an alkylate product;
(cc) separating the effluent of step (bb), thereby obtaining a hydrocarbon-rich phase and an composite ionic liquid-rich phase;
(dd) fractionating the hydrocarbon-rich phase of step (cc), thereby obtaining at least the alkylate product and a isoparaffin-comprising stream; and
(ee) recycling of the composite ionic liquid-rich phase of step (cc) to step (bb).

In step (aa) of the alkylation process according to the present invention a hydrocarbon mixture comprising at least an isoparaffin and an olefin is provided.

Preferably, the hydrocarbon mixture comprises at least isobutane and optionally isopentane, or a mixture thereof, as an isoparaffin. The hydrocarbon mixture preferably comprises at least an olefin comprising in the range of from 2 to 8 carbon atoms, more preferably of from 3 to 6 carbon atoms, even more preferably 4 or 5 carbon atoms. Examples of suitable olefins include, propene, 1-butene, 2-butene, isobutene, 1-pentene, 2-pentene, 2-methyl-1-butene, 3-methyl-1-butene, 2-methyl-2-butene.

Isoparaffins and olefins are supplied to the process in a molar ratio, which is preferably 1 or higher, and typically in the range of from 1:1 to 40:1, more preferably 1:1 to 20:1. In the case of a continuous process, excess isoparaffin can be recycled for reuse in the hydrocarbon mixture.

In step (bb) of the process according to the present invention the mixture of step (aa) is subjected to an alkylation reaction, wherein the hydrocarbon mixture is reacted with an composite ionic liquid according to the present invention to obtain an effluent comprising at least an alkylate product.

Accordingly, the hydrocarbon mixture is mixed in the reaction zone with the composite ionic liquid to form a reaction mixture to react under alkylation conditions.

Mixing of the hydrocarbon mixture and the composite ionic liquid may be done by any suitable means for mixing two or more liquids, including dynamic and static mixers. As the reaction progresses, the reaction mixture will comprise alkylate in addition to the hydrocarbon reactants (isoparaffins and olefins) and the composite ionic liquid.

The alkylation conditions (or process conditions) are those known in the art for this type of alkylation processes. Actual operational process conditions are for example dependent of the exact composition of the hydrocarbon mixture and composite ionic liquid, and the like.

The temperature in the alkylation reactor is preferably in the range of from −20 to 100° C., more preferably in the range of from 0 to 50° C. In any case the temperature must be high enough to ensure that the composite ionic liquid is in the liquid state.

To suppress vapour formation in the reactor, the process may be performed under pressure; preferably the pressure in the reactor is in the range of from 0.1 to 1.6 MPa.

Preferably, the composite ionic liquid-rich phase to hydrocarbon-rich phase volume ratio in the alkylation reaction zone is at least 0.5, preferably 0.9 more preferably at least 1. Preferably, the composite ionic liquid-rich phase to hydrocarbon-rich phase volume ratio in the reaction zone is in the range of from 1 to 10.

The hydrocarbon mixture may be contacted with the composite ionic liquid in any suitable alkylation reactor. The hydrocarbon mixture may be contacted with the composite ionic liquid in a batch-wise, a semi-continuous or continuous process. Reactors such as used in liquid acid catalysed alkylation can be used (see L. F. Albright, Ind. Eng. Res. 48 (2009)1409 and A. Corma and A. Martinez, Catal. Rev. 35 (1993) 483); alternatively the reactor is a loop reactor, optionally with multiple injection points for the hydrocarbon feed, optionally equipped with static mixers to ensure good contact between the hydrocarbon mixture and composite ionic liquid, optionally with cooling in between the injection points, optionally by applying cooling via partial vaporization of volatile hydrocarbon components (see Catal. Rev. 35 (1993) 483), optionally with an outlet outside the reaction zone (see WO2011/015636). In the prior art diagrams are available of alkylation process line-ups which are suitable for application in the process of this invention, e.g. in U.S. Pat. No. 7,285,698, Oil & Gas J., vol 104 (40) (2006) p 52-56 and Catal. Rev. 35 (1993) 483.

Optionally, the effluent of step (bb) is partly recycled to the reaction zone.

In step (cc) of the process according to the present invention, the effluent of step (bb) is separated to obtain a hydrocarbon-rich phase and a composite ionic liquid-rich phase.

Reference herein to a hydrocarbon-rich phase is to a phase comprising more than 90 weight % of hydrocarbons, based on the total moles of hydrocarbon and composite ionic liquid.

Reference herein to a composite ionic liquid-rich phase is to a phase comprising more than 90 weight % of composite ionic liquid, based on the total moles of hydrocarbon and composite ionic liquid.

Due to the low affinity of the ionic liquid for hydrocarbons and the difference in density between the hydrocarbons and the composite ionic liquid, the separation between the two phases is suitably done using for example well known settler means, wherein the hydrocarbons and composite ionic liquid separate into an upper predominantly hydrocarbon phase and lower predominantly composite ionic liquid phase or by using any other suitable liquid/liquid separator. Such liquid/liquid separators are known to the skilled person and include cyclone and centrifugal separators. Optionally, part of the hydrocarbon-rich phase of the step (cc) is recycled to step (aa).

In step (dd) of the process according to the present invention the hydrocarbon-rich phase of the step (cc) is fractionated to obtain at least an alkylate and a isoparaffin-comprising stream.

Suitably, the hydrocarbon-rich phase is treated and/or fractionated (e.g. by distillation) to retrieve the alkylate and optionally other components in the hydrocarbon phase, such as unreacted isoparaffin or n-paraffins. Preferably, such isoparaffin is at least partly reused to form part of the isoparaffin feed provided to the process. This may be done by recycling at least part of the isoparaffin, or a stream comprising isoparaffin obtained from the fractionation of the hydrocarbon-rich phase, and combining it with the isoparaffin feed to the process.

Typically, the alkylate obtained in step (dd) of the process according to the present invention comprises alkanes with a carbon number from C5 to C12, including dimethylhexanes and trimethylpentanes.

Preferably, the alkylate of step(dd) comprises from 50 to 85 wt. % of trimethylpentane, more preferably from 62 to 84 wt. %, most preferably from 78 to 84 wt. % trimethylpentane based on the total amount of alkylate of step (dd).

Suitably, the alkylate of step (dd) has a research octane number above 85, preferably above 90, more preferably above 95 and most preferably above 98.

In step (ee) of the process according to the present invention, the composite ionic-liquid rich phase of step (cc) is recycled to step (bb).

The composite ionic liquid rich phase of step (cc) is generally recycled back to the reactor.

During the process to prepare an alkylate according to the present invention solids may be formed.

Reference herein to solids is to non-dissolved solid particles. The solids predominantly consist of metals, metal compounds and/or metal salts which were originally comprised in the composite ionic liquid catalyst. Typically, the solids comprise at least 10 wt % metal, i.e. either in metallic, covalently bound or ionic form, based the total weight of the solids, wherein the metal is a metal that was introduced to the process as part of the acidic ionic liquid catalyst. The solids may also comprise contaminant components, which were introduced into the reaction mixture as contaminants in the hydrocarbon mixture or the composite ionic liquid. Alternatively, (part of) the solids may be the product of a chemical reaction involving any of the above-mentioned compounds, e.g. polymeric substances.

The solids may have any size, however the solids typically have an average size of in the range of from 0.1 to 10 μm. In particular, at least 50% of the solids have a particle size below 5 μm, more particular 80% of the solids have a particle size below 5 μm based on the total number of solid particles.

The advantage of the used of the composite ionic liquid obtainable by the present invention to prepare an alkylate is that the formation of solids during alkylation is reduced.

The invention is illustrated by the following non-limiting examples.

### Example 1.1 Preparation of Et₃NHCl Composite IL 1.8AlCl₃-0.5CuCl (IL-1)

Et₃NHCl and AlCl₃ are commercially obtained from Aladdin Industrial Inc.

137.65 g of Et₃NHCl (1 mol) was placed in a 500 mL flask under N₂ atmosphere. Subsequently, 133.34 g of AlCl₃ (1 mol) was added into the flask. A reaction started and the mixture was stirred while the temperature raised to 100° C. by the exothermic reaction. When the temperature had decreased below 60° C. by cooling to the atmosphere 50 g of CuCl (0.5 mol) was added to the IL mixture. The IL mixture was heated as soon as the temperature started to drop and kept at 120° C. for at least 2 hours by external heating. Then another portion of 106.67 g of AlCl₃ (0.8 mol) was added into the flask. The temperature of IL rose to 150° C. The temperature of mixture was kept at 150° C. for at least 4 hours using external heating, after which the composite IL (417 g) was allowed to cool down to room temperature.

### Example 1.2 Preparation of Et₃NHCl Composite IL 1.8AlCl₃-0.4CuCl (IL-2)

The procedure of example 1 was repeated using 40 g of CuCl (0.4 mol) instead of 50 g of CuCl (0.5 mol). 417 g of IL-2 was obtained.

### Example 1.3 Preparation of Et₃NHCl Composite IL 1.8AlCl₃-0.3CuCl (IL-3)

The procedure of example 1 was repeated using 30 g of CuCl (0.3 mol) instead of 50 g of CuCl (0.5 mol). 407 g of IL-3 was obtained.

### Example 1.4 Preparation of Et₃NHCl Composite IL 1.8AlCl₃-0.2CuCl (IL-3)

The procedure of example 1 was repeated using 20 g of CuCl (0.2 mol) instead of 50 g of CuCl (0.5 mol). 397 g of IL-4 was obtained.

### Example 2 the Addition of Hydrocarbon Coker Gasoline

### Example 2.1 Preparation of Et₃NHCl Composite IL 1.8AlCl₃-0.4CuCl (IL-5)

Coker gasoline is commercially obtained from SINOPEC Beijing Yanshan Company.

137.65 g of Et₃NHCl (1 mol) was placed in a 500 mL flask under N₂ atmosphere. Subsequently, 133.34 g of AlCl₃ (1 mol) was added into the flask. A reaction started and the mixture was stirred while the temperature raised to 100° C. by the exothermic reaction. When the temperature had decreased below 60° C. by cooling to the atmosphere 40 g of CuCl (0.4 mol) was added to the IL mixture. The IL mixture was heated as soon as the temperature started to drop and kept at 120° C. for at least 2 hours by external heating. Then another portion of 106.67 g of AlCl₃ (0.8 mol) was added into the flask. The temperature of IL rose to 150° C. The temperature of mixture was kept at 150° C. for at least 4 hours using external heating.

Then 5 mL of coker gasoline was added into the flask. The IL mixture was heated as soon as the temperature started to drop and kept at 150° C. for at least 1 hour using external heating, after which the composite IL (418 g) was allowed to cool down to room temperature.

### Example 2.2 Preparation of Et₃NHCl Composite IL 1.8AlCl₃-0.3CuCl (IL-6)

The procedure of example 2.1 was repeated using 30 g of CuCl (0.3 mol) instead of 40 g of CuCl (0.4 mol). 408 g of IL-6 was obtained.

### Example 2.3 Preparation of Et₃NHCl Composite IL 1.8AlCl₃-0.2CuCl (IL-7)

The procedure of example 2.1 was repeated using 20 g of CuCl (0.2 mol) instead of 40 g of CuCl (0.4 mol). 398 g of IL-7 was obtained.

### Example 3 Addition of Hydrocarbon Toluene

### Example 3.1 Preparation of Et₃NHCl Composite IL 1.8AlCl₃-0.4 CuCl (IL-8)

Toluene is commercially obtained from Aladdin Industrial Inc.

137.65 g of Et₃NHCl (1 mol) was placed in a 500 mL flask under N₂ atmosphere. Subsequently, 133.34 g of AlCl₃ (1 mol) was added into the flask. A reaction started and the mixture was stirred while the temperature raised to 100° C. by the exothermic reaction. When the temperature had decreased below 60° C. by cooling to the atmosphere 40 g of CuCl (0.4 mol) was added to the IL mixture. The IL mixture was heated as soon as the temperature started to drop and kept at 120° C. for at least 2 hours by external heating. Then another portion of 106.67 g AlCl₃ (0.8 mol) was added into the flask. The temperature of IL rose to 150° C. The temperature of mixture was kept at 150° C. for at least 2 hours using external heating.

Then 1 g (1.15 mL) of toluene was added into the flask. The IL mixture was heated as soon as the temperature started to drop and kept at 150° C. for at least 2 hours using external heating, after which the composite IL (417 g) was allowed to cool down to room temperature.

### Example 3.2 Preparation of Et₃NHCl Composite IL 1.8AlCl₃-0.4 CuCl (IL-9)

The procedure of example 3.1 was repeated using 3 g (3.45 mL) of toluene instead of 1 g (1.15 mL) of toluene. 418 g IL-9 was obtained.

### Example 3.3 Preparation of Et₃NHCl Composite IL 1.8AlCl₃-0.3 CuCl (IL-10)

The procedure of example 3.1 was repeated using 30 g of CuCl (0.3 mol) instead of 40 g of CuCl (0.4 mol). 407 g of IL-10 was obtained.

### Example 3.4 Preparation of Et₃NHCl Composite IL 1.8AlCl₃-0.3 CuCl (IL-11)

The procedure of example 3.3 was repeated using 2 g (2.3 mL) of toluene instead of 1 g (1.15 mL) of toluene. 408 g of IL-11 was obtained.

### Example 3.5 Preparation of Et₃NHCl Composite IL 1.8AlCl₃-0.2 CuCl (IL-12)

The procedure of example 3.1 was repeated using 20 g of CuCl (0.2 mol) instead of 40 g of CuCl (0.4 mol). 397 g of IL-12 was obtained.

### Example 3.6 Preparation of Et₃NHCl Composite IL 1.8AlCl₃-0.2 CuCl (IL-13)

The procedure of example 3.5 was repeated using 2 g (2.3 mL) of toluene instead of 1 g (1.15 mL) of toluene. 398 g of IL-13 was obtained.

### Example 4 Alkylation Tests with IL-1-IL-13

200 g of composite IL-10 was placed into a 500 mL autoclave. The autoclave was closed, the stirrer was started, and the temperature inside the autoclave was controlled at 20° C. C4 feed with an I/O ratio (isobutane/2-butene) of 20 mol/mol was pumped through a filter and a dryer, and then entered into the autoclave. The feed rate was controlled at 700 mL/h by the plunger pump. The pressure in the autoclave was maintained at 0.6 MPa to keep the reactants and product in liquid phase. During reaction and filling the autoclave, the reaction system was separating into two phases due to the differences in density. The upper part of the reaction mixture in the autoclave was the unreacted feed and products, while the lower part consisted of a mixture of ionic liquid and hydrocarbons. When the autoclave was filled completely a sample was taken from the overflow of the autoclave.

Analysis of Feed and Products of Alkylation Tests with IL-1-IL-13

Hydrocarbon Composition of Feed:

The C4 feed (gas sample) was analyzed by an Agilent refinery gas analyzer (an Agilent 6890 gas chromatograph with Chem Station software) to determine the volume percentage of the components. Data were converted to mass percentages with the state equation of ideal gases. The water content of the C4 feed was measured by Karl-Fisher analyzer.

Hydrocarbon Composition of Alkylate Product:

The alkylate products were analyzed by a GC SP3420, equipped with a flame ionization detector (FID). The components in the product were separated by a 50 m PONA capillary column (ID 0.25 mm, 0.25 μm film thickness). The temperatures of injector and detector were 250° C. and 300° C., respectively. The temperature program was as follows, holding at 40° C. for two minutes, increasing to 60° C. at a speed of 2° C./min, increasing to 120° C. at a speed of 1° C./min, increasing to 180° C. at a speed of 2° C./min, and finally holding at 180° C. for thirteen minutes. The hydrocarbons were identified by their retention time and quantitative analysis was done by their normalised areas.

The RON of alkylate was calculated according to the equation (1).

$$RON = {\sum }_{i = 1}^{n} {C}_{i} \cdot {RON}_{i} \qquad ( 1 )$$

In this equation, i is a component in alkylate, Cᵢ is the relative content of component i in alkylate, wt %, RONᵢ is the RON of component i.

Results from Alkylation Tests

TABLE 1
Composition of alkylate catalyzed by IL-1 to IL-13

| | | Vol- | | | | | | |
|---|---|---|---|---|---|---|---|---|
| | Molar ratio | ume/ | C5- | | | | | |
| | of Et₃NHCl/ | mass | C7 | TMP | DMH | | C9⁺ | |
| Cat. | AlCl₃/CuCl | of HC | wt % | wt % | wt % | T/D | wt % | RON |
| IL-1 | 1.0/1.8/0.5 | 0 | 8.1 | 82.0 | 6.8 | 12.1 | 3.1 | 97.3 |
| IL-2 | 1.0/1.8/0.4 | 0 | 11.4 | 68.5 | 10.4 | 6.7 | 9.7 | 93.2 |
| IL-3 | 1.0/1.8/0.3 | 0 | 13.5 | 62.3 | 13.3 | 4.7 | 10.9 | 91.5 |
| IL-4 | 1.0/1.8/0.2 | 0 | 16.4 | 52.5 | 18.6 | 2.8 | 12.5 | 86.5 |
| IL-5 | 1.0/1.8/0.4 | 5 mL of | 7.3 | 83.4 | 6.7 | 12.4 | 1.9 | 98.1 |
| | | Coker | | | | | | |
| | | gasoline | | | | | | |
| IL-6 | 1.0/1.8/0.3 | 5 mL of | 8.3 | 78.2 | 8.5 | 9.2 | 5.0 | 96.5 |
| | | Coker | | | | | | |
| | | gasoline | | | | | | |
| IL-7 | 1.0/1.8/0.2 | 5 mL of | 11.3 | 70.0 | 11.7 | 6.0 | 6.9 | 93.8 |
| | | Coker | | | | | | |
| | | gasoline | | | | | | |
| IL-8 | 1.0/1.8/0.4 | 1 g of | 11.9 | 70.1 | 9.9 | 7.1 | 8.1 | 94.2 |
| | | toluene | | | | | | |
| IL-9 | 1.0/1.8/0.4 | 2 g of | 9.4 | 82.7 | 5.9 | 14.0 | 2.0 | 98.0 |
| | | toluene | | | | | | |
| IL-10 | 1.0/1.8/0.3 | 1 g of | 12.0 | 68.4 | 11.4 | 6.0 | 8.1 | 93.2 |
| | | toluene | | | | | | |
| IL-11 | 1.0/1.8/0.3 | 2 g of | 9.1 | 76.8 | 7.7 | 10.0 | 6.4 | 95.3 |
| | | toluene | | | | | | |
| IL-12 | 1.0/1.8/0.2 | 1 g of | 15.7 | 60.1 | 12.2 | 4.9 | 12.0 | 89.0 |
| | | toluene | | | | | | |
| IL-13 | 1.0/1.8/0.2 | 2 g of | 14.5 | 62.6 | 11.6 | 5.4 | 11.0 | 91.3 |
| | | toluene | | | | | | |

### DISCUSSION

Table 1 shows that the use of less CuCl in Et₃NHCl composite ionic liquid (see Table 1, IL-2-IL-4) results in lower selectivity to TMP and RON of alkylate than when a Et₃NHCl composite liquid comprising a higher amount of CulCl (see Table 1, IL-1) is used.

The results in Table 1 also shows that by partly replacing CuCl in the composite ionic liquid synthesis process by hydrocarbons coker gasoline (see Table 1 IL-5-IL8) and by toluene (see Table 1, IL-9-IL-11) the selectivity to TMP and RON of alkylate produced by Et₃NHCl composite ionic liquid comprising a hydrocarbon, which has partly replaced CuCl in the ionic liquid, is higher than the composite ionic liquids which were prepared without using those hydrocarbons in their preparation process (see IL-1 to IL-4).

The composite ionic liquids prepared with the addition of the hydrocarbons coker gasoline and toluene results in a higher selectivity to TMP and RON of alkylate than the Et₃NHCl composite ionic liquid comprising a high amount of CuCl (See Table 1, IL-5 and IL-9 versus to IL-1).

1. A process to prepare a composite ionic liquid, the process at least comprising the steps:
(a) mixing an ammonium salt and a solid aluminium salt to obtain a first mixture;
(b) stirring under heating the first mixture of step (a);
(c) adding to the first mixture of step (b) one or more solid metal salts to obtain a second mixture, wherein the metal salts are selected from halides, sulfates, or nitrates of aluminium, gallium, copper, iron, zinc, nickel, cobalt, molybdenum and platinum;
(d) stirring under heating the second mixture of step (c);
(e) adding to the second mixture of step (d) a hydrocarbon to obtain a third mixture;
(f) stirring under heating the third mixture of step (e) until the solids of the aluminium salt of step (a), and the solids of the metal salts of step (c) disappear and the mixture is converted into a composite ionic liquid; and
(g) cooling the composite ionic liquid of step (f).

2. The process according to claim 1, wherein the ammonium salt in step (a) is a hydrohalide of an alkyl-containing amine, preferably triethylammonium hydrogenchloride (Et₃NHCl).

3. The process according to claim 1, wherein the aluminium salt in step (a) is an aluminium halide, preferably aluminium (III) chloride.

4. The process according to claim 1, wherein the molar ratio of the aluminium salt to the ammonium salt in the composite ionic liquid is from 1.2 to 2.2.

5. The process according to claim 1, wherein the molar ratio of the aluminium (III) chloride to Et₃NHCl in the composite ionic liquid is from 1.2 to 2.2.

6. The process according to claim 1, wherein the solid metal salts added to the first mixture in step (c) are halides of aluminium and copper.

7. The process according to claim 6, wherein to the first mixture in step (c) aluminium(III)chloride and copper(I)chloride are added.

8. The process according to claim 1, wherein the molar ratio of the metal halide of step (c) to the ammonium salt of step (a) in the composite ionic liquid is from 0.1 to 0.5.

9. The process according to claim 1, wherein the molar ratio of the copper(I)chloride to the Et₃NHCl in the composite ionic liquid is from 0.1 to 0.5.

10. The process according to claim 1, wherein the amount of hydrocarbon added to the second mixture in step (e) is in the range of 0.5 to 10 mL per 1 mol of ammonium salt.

11. The process according to, wherein the amount of hydrocarbon added to the second mixture in step (e) is in the range of 0.5 to 10 mL per 1 mol of Et₃NHCl.

12. The process according to claim 1, wherein the hydrocarbon added to the second mixture in step (e) is toluene or coker gasoline.

13. A composite ionic liquid that has been obtained according to claim 1.

14. The process to prepare an alkylate product, the process at least comprising the steps:
(aa) providing a hydrocarbon mixture comprising at least an isoparaffin and an olefin;
(bb) subjecting the mixture of step (aa) to an alkylation reaction between the isoparaffin and the olefin, wherein the hydrocarbon mixture is reacted with a composite ionic liquid as claimed in claim 13 to obtain an effluent comprising at least an alkylate product;
(cc) separating the effluent of step (bb), thereby obtaining a hydrocarbon-rich phase and an composite ionic liquid-rich phase;
(dd) fractionating the hydrocarbon-rich phase of step (cc), thereby obtaining at least the alkylate product and a isoparaffin-comprising stream; and
(ee) recycling of the composite ionic liquid-rich phase of step (cc) to step (bb)

15. The process according to claim 14, wherein the alkylate of step (dd) comprises from 50 to 85 wt. %, based on the total amount of alkylate of step (dd).
